(19) United States
(12) Patent Application Publication
(10) Pub. No.: US 2025/0032256 A1
(43) Pub. Date: Jan. 30, 2025

(54) SUPPORT DEVICE FOR VALVE LEAFLET

(71) Applicant: EDWARDS LIFESCIENCES CORPORATION, Irvine, CA (US)

(72) Inventors: Felino V. Cortez, JR., Bowie, MD (US); Stephen Cournane, Severn, MD (US); Nancy Ling Chung, Edgewater, MD (US)

(21) Appl. No.: 18/914,498

(22) Filed: Oct. 14, 2024

Related U.S. Application Data
(63) Continuation of application No. PCT/US2023/017640, filed on Apr. 5, 2023.
(60) Provisional application No. 63/362,995, filed on Apr. 14, 2022.

(51) Int. Cl. A61F 2/24; G09B 23/30

(52) U.S. Cl. CPC A61F 2/2457; A61F 2/2466; G09B 23/30

(57) ABSTRACT

A delivery system comprises a first delivery shaft configured to deliver one or more sutures to a leaflet of a heart. The first delivery shaft has an end effector at a distal end of the first delivery shaft and configured to contact the leaflet. The delivery system further comprises a needle configured to extend from the distal end of the first delivery shaft and through the leaflet and a support arm configured to contact and extend at least partially along the leaflet during delivery of the needle through the leaflet.

### CROSS REFERENCE TO RELATED APPLICATIONS

This application is a continuation of International Patent Application No. PCT/US2023/017640, filed Apr. 5, 2023, which claims the benefit of U.S. Patent Application No. 63/362,995, filed Apr. 14, 2022, the entire disclosures all of which are incorporated by reference for all purposes.

### BACKGROUND

### Technical Field

The disclosure herein relates to cardiac valve repairs, and more particularly to surgical suture placement in connection with minimally invasive valve repair operations.

### Description of Related Art

Certain medical and other procedures involve the use of sutures or other similar devices.

### SUMMARY

In accordance with some implementations of the present disclosure, a delivery system comprises a first delivery shaft configured to deliver one or more sutures to a leaflet of a heart. The first delivery shaft has an end effector at a distal end of the first delivery shaft and configured to contact the leaflet. The delivery system further comprises a needle configured to extend from the distal end of the first delivery shaft and through the leaflet and a first support arm configured to contact and extend at least partially along the leaflet during delivery of the needle through the leaflet.

In some examples, each of the one or more sutures is wrapped at least partially around a distal portion of the needle and is configured to form a knot anchor when removed from the needle. The first support arm may be configured to extend at least partially along an underside of the leaflet.

The delivery system may further comprise a second support arm configured to extend at least partially along an upper side of the leaflet. In some examples, the first support arm is configured to extend at least partially along an upper side of the leaflet.

In some examples, the delivery system further comprises a second support arm configured to extend at least partially along an underside of the leaflet. The first support arm may be configured to press down on the leaflet while the second support arm presses up on the leaflet.

The first support arm may be configured to press down on the leaflet while the end effector presses up on the leaflet. In some examples, the delivery system further comprises a support shaft configured to deliver the first support arm.

In some examples, the end effector is configured to contact an underside of the leaflet. The end effector may be configured to contact an upper side of the leaflet.

The one or more sutures may be configured to form loops extending at least partially through the leaflet. In some examples, the first support arm forms a loop with an opening.

In some examples, the needle is configured to extend through the opening. The first support arm may comprise a sheet supported by a wireframe. A distal portion of the sheet may have a circular shape.

A distal portion of the first support arm may be configured to extend generally perpendicularly relative to a proximal portion of the first support arm. In some examples, the distal portion is configured to naturally extend generally perpendicularly in response to removal from a support shaft.

In some examples, the first support arm has a braided wire form. The first delivery shaft may comprise a distal portion configured to rotate independently of a proximal portion of the first delivery shaft.

Some implementations of the present disclosure relate to a method comprising delivering a first delivery shaft to an underside of a leaflet of a heart, the first delivery shaft having an end effector at a distal end of the first delivery shaft. The first delivery shaft carries a needle and one or more sutures. The method further comprises extending a first support arm along the leaflet, sliding the end effector towards an edge portion of the leaflet, and extending the needle through the leaflet at the edge portion while the first support arm and the end effector are in contact with the leaflet.

Each of the one or more sutures may be wrapped at least partially around a distal portion of the needle and is configured to form a knot anchor when removed from the needle. In some examples, the method further comprises extending the first support arm at least partially along an underside of the leaflet.

In some examples, the method further comprises extending a second support arm at least partially along an upper side of the leaflet. The method may further comprise delivering a support shaft carrying the first support arm to the underside of the leaflet.

The method may further comprise extending the first support arm at least partially above the leaflet. In some examples, the method further comprises lowering the first support arm to contact an upper side of the leaflet.

In some examples, the needle is extended through the leaflet while the leaflet is at least partially immobilized between the first support arm and the end effector. The needle may be extended through an opening of the first support arm above the leaflet.

The method may further comprise pressing the first support arm downward against the leaflet while pressing the end effector upward against the leaflet. In some examples, the method further comprises extending a second support arm at least partially below the leaflet.

In some examples, the method further comprises rotating a distal portion of the first delivery shaft relative to a proximal portion of the first delivery shaft to position the first support arm.

The one or more sutures may be configured to form loops extending at least partially through the leaflet. In some examples, the first support arm comprises a sheet supported by a wireframe.

In some examples, a distal portion of the sheet has a circular shape. A distal portion of the first support arm may be configured to extend generally perpendicularly relative to a proximal portion of the first support arm. The distal portion may be configured to naturally extend generally perpendicularly in response to removal from a support shaft. The first support arm may have a braided wire form.

All methods disclosed herein also encompass simulations of the methods, for example, for training; testing; demonstration; or device or procedure development. Methods for treating a patient can include simulating treatment on a simulated human or non-human patient, for example, an anthropomorphic ghost. Examples of suitable simulated patients can include both an entire body, any portion of a body, or at least a portion of an organ, for example, a heart. The simulations can be physical, virtual, or any combination thereof. Examples of physical simulations can include any combination of natural or manufactured whole human or animal cadavers, portions thereof, or cadaver organs. Virtual simulations can include any combination of virtual reality, projections onto a screen or on at least a portion of a physical simulation, or other in silico elements. Some simulations can include non-visual elements, for example, auditory, tactile, or olfactory stimuli.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 is a cutaway view of the human heart.

FIG. 2 is a perspective view of a tissue anchor delivery device in accordance with one or more examples.

FIGS. 3A-3D illustrate a delivery system for delivering an example support device via a shaft in accordance with one or more examples.

FIGS. 4A-4C illustrate a cutaway view of a delivery system for delivering one or more suture anchors and/or support devices at or near one or more valve leaflets of a heart in accordance with one or more examples.

FIGS. 5A and 5B illustrate delivery of one or more suture anchors via an example delivery system through one or more leaflets of a heart valve in accordance with one or more examples.

FIG. 6 (FIGS. 6-1 and 6-2) is a flow diagram illustrating a process for delivering one or more leaflet anchors to a valve leaflet in accordance with one or more examples of the present disclosure.

FIG. 7 (FIGS. 7-1 and 7-2) provides illustrations corresponding to steps of the process described in FIG. 6.

FIG. 7 shows a cutaway view of a sutureform deployed from the anchor delivery device in a heart in accordance with one or more examples.

FIG. 8 illustrates a cutaway view of a delivery system for delivering one or more suture anchors and/or support devices at or near one or more valve leaflets of a heart in accordance with one or more examples.

FIG. 9 illustrates an example support shaft for delivering one or more support devices in accordance with one or more examples.

FIG. 10 (FIGS. 10-1, 10-2, and 10-3) is a flow diagram illustrating a process for delivering one or more leaflet anchors to a valve leaflet in accordance with one or more examples of the present disclosure.

FIG. 11 (FIGS. 11-1, 11-2, and 11-3) provides illustrations corresponding to steps of the process described in FIG. 10.

To further clarify various aspects of examples of the present disclosure, a more particular description of certain examples will be made by reference to various aspects of the appended drawings. It is appreciated that these drawings depict only typical examples of the present disclosure and are therefore not to be considered limiting of the scope of the disclosure. Moreover, while the figures can be drawn to scale for some examples, the figures are not necessarily drawn to scale for all examples. Examples of the present disclosure will be described and explained with additional specificity and detail through the use of the accompanying drawings.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

The following description refers to the accompanying drawings, which illustrate specific examples. Other examples having different structures and operation do not depart from the scope of the disclosure.

### Overview

Examples of the present disclosure provide solutions relating to the treatment of certain structural heart conditions with multiple valve leaflet anchors and/or devices configured to deliver multiple valve leaflet anchors. Various disease processes can impair the proper functioning of one or more of the valves of the heart. These disease processes include degenerative processes (e.g., Barlow's disease, fibroelastic deficiency), inflammatory processes (e.g., rheumatic heart disease), and infectious processes (e.g., endocarditis). Additionally, damage to the ventricle from prior heart attacks (e.g., myocardial infarction secondary to coronary artery disease) or other heart diseases (e.g., cardiomyopathy) can distort the geometry of the heart causing valves in the heart to dysfunction. Many patients undergoing valve surgery, such as mitral valve surgery, suffer from a degenerative disease that causes a malfunction in a leaflet of the valve, which results in prolapse and regurgitation.

Valve regurgitation occurs when the leaflets of the valve do not close completely, thereby allowing blood to leak back into the prior chamber when the heart contracts. There are generally three mechanisms by which a valve becomes regurgitant or incompetent, including Carpentier's type I, type II and type III malfunctions. A Carpentier type I malfunction involves the dilation of the annulus such that the area of the valve orifice increases. The otherwise normally functioning leaflets do not have enough surface area to cover the enlarged orifice and fail to form a tight seal (e.g., do not coapt properly) causing regurgitation. Included in a type I mechanism malfunction are perforations of the valve leaflets, as in endocarditis. A Carpentier's type II malfunction involves prolapse of a segment of one or both leaflets above the plane of coaptation. This is the most commonly treated cause of mitral regurgitation, and is often caused by the stretching or rupturing of chordae tendineae normally connected to the leaflet. A Carpentier's type III malfunction involves restriction of the motion of one or more leaflets such that the leaflets are abnormally constrained below the level of the plane of the annulus. Leaflet restriction can be caused by rheumatic heart disease (IIIa) or dilation of the ventricle (IIIb).

Mitral valve disease is the most common valvular heart disorder, with nearly 4 million Americans estimated to have moderate to severe mitral valve regurgitation (“MR”), with similar numbers of individuals impacted outside of the United States. MR can result in a volume overload on the left ventricle which in turn progresses to ventricular dilation, decreased ejection performance, pulmonary hypertension, symptomatic congestive heart failure, atrial fibrillation, right ventricular dysfunction, and/or death. Successful surgical mitral valve repair can at least partially restore mitral valve competence, abolish the volume overload on the left ventricle, improve symptom status, and/or prevent adverse left ventricular remodeling. While generally safe and effective, conventional open-heart operations are invasive, result in significant disability, and require extended post-procedure recovery. Patients routinely spend five to seven days in the hospital and often are not able to return to normal daily activities for a month or more.

In many examples of mitral valve regurgitation, repair may be preferable to valve replacement. There are a variety of advantages to performing heart valve repair (e.g., mitral valve repair) using less invasive procedures while the heart is still beating, as described in detail herein. Mitral valve repair procedures may rely upon use of visualization technology, such as sonic guidance, which may have limitations that can reduce the effectiveness of such repairs. Accordingly, there is a continuing need for new procedures and devices for performing less invasive mitral valve repairs which do not require cardiac arrest and are less technologically challenging.

In some implementations, the present disclosure relates to an anchor delivery system configured to deliver multiple suture knots simultaneously, near-simultaneously, and/or during a single deployment stage. The term “suture” is used herein according to its plain and ordinary meaning and may refer to any elongate cord strip, strand, line, tie, string, ribbon, strap, or portion thereof, or other type of material used in medical procedures. One having ordinary skill in the art will understand that a wire or other similar material may be used in place of a suture. Furthermore, in some contexts herein, the terms “cord” and “suture” may be used substantially interchangeably. In addition, use of the singular form of any of the suture-related terms listed above, including the terms “suture” and “cord,” may be used to refer to a single suture/cord, or to a portion thereof. For example, where a suture knot or anchor is deployed on a distal side of a tissue portion, and where two suture portions extend from the knot/anchor on a proximal side of the tissue, either of the suture portions may be referred to as a “suture” or a “cord,” regardless of whether both portions are part of a unitary suture or cord.

Certain inventive features disclosed herein relate to the delivery of sutures and/or suture knots associated with certain heart valve repair systems and devices, and/or systems, process, and devices for repairing any other type of target organ tissue. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, or otherwise physically related to the second feature, element, component, device, or member.

When anchoring and/or attaching sutures to valve leaflets, it may be beneficial to anchor and/or attach at an edge portion (e.g., at or near a commissure of the valve) of the leaflet. For example, artificial chords anchored to a valve leaflet may have an improved effect on the leaflet when anchored at or near an edge portion. However, it can be difficult to anchor and/or attach a suture at or near the edge portion due at least in part to a lack of stability of the leaflet and/or a risk of delivery devices slipping off of the leaflet during an anchoring process.

Examples described herein may advantageously relate to devices and/or processes involving a retractable support arm that can be used to support and/or immobilize valve leaflets during delivery of one or more sutures (e.g., suture anchors and/or suture loops) at the leaflets. The term “support arm” is used herein in accordance with its plain and ordinary meaning and may refer to any elongate member, protrusion, extension, beam, platform, bar, wireframe, wire, sheet, loop, lasso, hoop, and/or other device configured to extend along an underside and/or upper side of a valve leaflet and/or to support a valve leaflet. A support arm may include any means for supporting a valve leaflet from an upper side of the leaflet and/or an underside of the leaflet. In some examples, a support arm may not penetrate and/or puncture a leaflet. However, in alternative examples a support arm may pierce and/or otherwise anchor to a valve leaflet. In some examples, a support arm may comprise one or more clips, fasteners, hooks, and/or other features to grip onto a valve leaflet. Moreover, a support arm may comprise various features (e.g., teeth, grooves, etc.) to improve a grip of the support arm on a valve leaflet.

In some examples, a support arm may be used in combination with an end effector and/or a catheter comprising an end effector at a distal portion of the catheter. Use of one or more support arms can allow for use of an end effector with a minimal profile. For example, the end effector may not be required to have a relatively large profile because of added support provided by the one or more support arms. Support arms can advantageously allow for implantation of artificial cords/chords at or near an edge of a leaflet, increase stability of a delivery device during actuation, prevent a distal tip of a delivery device (e.g., a catheter) from slipping off the leaflet edge, and/or improve targeting of medical implants. While some examples described herein relate to use of one or more support arms during delivery of one or more suture anchors wrapped along a needle, support arms may additionally or alternatively be used in other processes, including transcatheter valve repair.

In some examples, a support arm may be at least partially composed of one or more shape memory materials (e.g., nitinol). Support arms may be at least partially expandable to support a large area of a leaflet. In some examples, a support arm may be contoured to a leaflet. For example, a support arm can have a convex and/or concave form to approximate a curvature of a valve leaflet (e.g., a mitral valve leaflet). In some examples, a support arm can be echogenic and/or radiopaque to facilitate various imaging technologies (e.g., ultrasound and/or computed tomography (CT) imaging).

The following includes a general description of human cardiac anatomy that is relevant to certain inventive features and examples disclosed herein and is included to provide context for certain aspects of the present disclosure. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

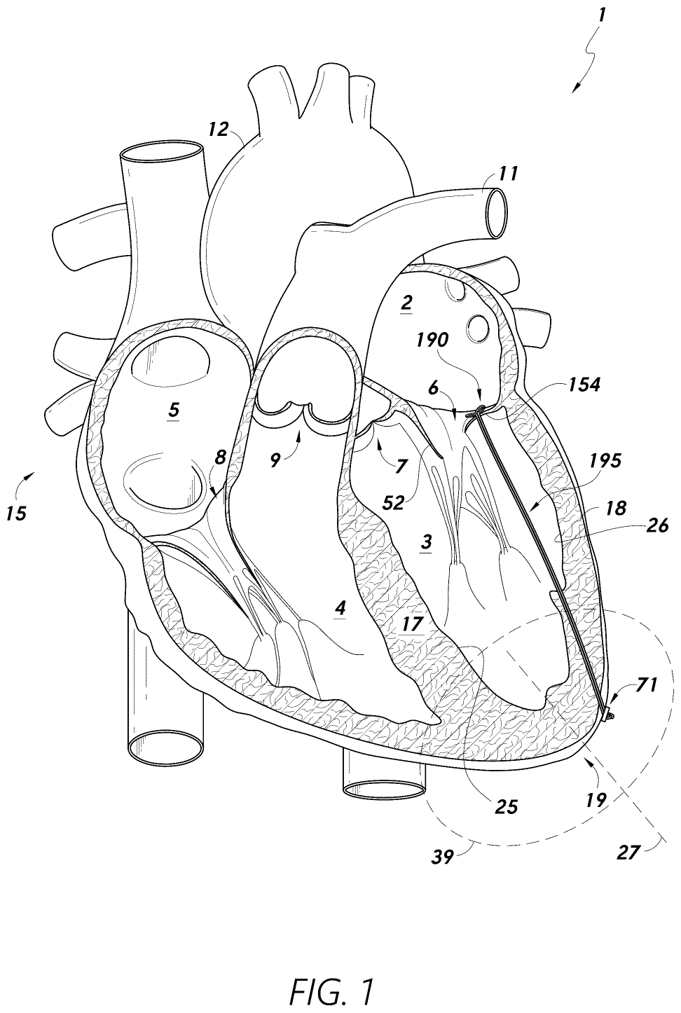

FIG. 1 illustrates an example representation of a heart 1 having various features relevant to certain aspects of the present inventive disclosure. The heart 1 includes four chambers, namely the left ventricle 3, the left atrium 2, the right ventricle 4, and the right atrium 5. A wall of muscle, referred to as the septum 17, separates the left 2 and right 5 atria and the left 3 and right 4 ventricles. The inferior tip 19 of the heart 1 is referred to as the “apex” 19 and is generally located on the midclavicular line, in the fifth intercostal space. The apex 19 can be considered part of the greater apical region 39.

The left ventricle 3 is the primary pumping chamber of the heart 1. A healthy left ventricle is generally conical and/or apical in shape in that it is longer (along a longitudinal axis extending in a direction from the aortic valve 7 to the apex 19) than it is wide (along a transverse axis extending between opposing walls 25, 26 at the widest point of the left ventricle) and descends from a base (including the left ventricle papillary muscles 15) with a decreasing cross-sectional circumference to the point or apex 19. Generally, the apical region 39 of the heart is a bottom region of the heart that is within the left or right ventricular region but is distal to the mitral 6 and tricuspid 8 valves and toward the tip of the heart. More specifically, the apical region 39 may be considered to be within about 20 cm to the right or to the left of the median axis 27 of the heart 1.

The pumping of blood from the left ventricle is accomplished by a squeezing motion and a twisting or torsional motion. The squeezing motion occurs between the lateral wall 18 of the left ventricle and the septum 17. The twisting motion is a result of heart muscle fibers that extend in a circular or spiral direction around the heart. When these fibers contract, they produce a gradient of angular displacements of the myocardium from the apex 19 to the base about the longitudinal axis of the heart. The resultant force vectors extend at angles from about 30-60 degrees to the flow of blood through the aortic valve 7. The contraction of the heart is manifested as a counterclockwise rotation of the apex 19 relative to the base, when viewed from the apex 19. A healthy heart can pump blood from the left ventricle in a very efficient manner due to the spiral contractility of the heart.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (e.g., systole) and open during ventricular expansion (e.g., diastole). The valves of the heart 1 further include the pulmonary valve 9, which separates the right ventricle 4 from the pulmonary artery 11 and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The atrioventricular (e.g., mitral and tricuspid) heart valves may comprise a collection of chordae tendineae (13, 16) and papillary muscles (10, 15) for securing the leaflets of the respective valves to promote and/or facilitate proper coaptation of the valve leaflets and prevent prolapse thereof. The papillary muscles, for example, may generally comprise finger-like projections from the ventricle wall. With respect to the tricuspid valve 8, the normal tricuspid valve may comprise three leaflets and three corresponding papillary muscles 10 (two shown in FIG. 1). The leaflets of the tricuspid valve may be referred to as the anterior, posterior and septal leaflets, respectively. The valve leaflets are connected to the papillary muscles 10 by the chordae tendineae 13, which are disposed in the right ventricle 4 along with the papillary muscles 10.

Surrounding the ventricles (3, 4) are a number of arteries (not shown) that supply oxygenated blood to the heart muscle and a number of veins that return the blood from the heart muscle. The coronary sinus (not shown) is a relatively large vein that extends generally around the upper portion of the left ventricle 3 and provides a return conduit for blood returning to the right atrium 5. The coronary sinus terminates at the coronary ostium (not shown) through which the blood enters the right atrium.

With respect to the mitral valve 6, a normal mitral valve may comprise two leaflets (anterior and posterior) and two corresponding papillary muscles 15. The papillary muscles 15 originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies.

Various disease processes can impair the proper functioning of one or more of the valves of the heart. These disease processes include degenerative processes (e.g., Barlow's disease, fibroelastic deficiency), inflammatory processes (e.g., rheumatic heart disease) and infectious processes (e.g., endocarditis). Additionally, damage to the ventricle from prior heart attacks (e.g., myocardial infarction secondary to coronary artery disease) or other heart diseases (e.g., cardiomyopathy) can distort the valve's geometry causing it to dysfunction. However, the vast majority of patients undergoing valve surgery, such as mitral valve surgery, suffer from a degenerative disease that causes a malfunction in one or more leaflets of the valve which results in prolapse and regurgitation.

The mitral valve 6 and tricuspid valve 8 can be divided into three parts: an annulus, leaflets, and a sub-valvular apparatus. The sub-valvular apparatus can be considered to include the papillary muscles 10, 15 and the chordae tendineae 13, 16, which can elongate and/or rupture. If a valve is functioning properly, when closed, the free margins or edges of the leaflets come together and form a tight junction, the are of which, in the mitral valve, is known as the line, plane or area of coaptation. Normal mitral and tricuspid valves open when the ventricles relax allowing blood from the atrium to fill the decompressed ventricle. When the ventricle contracts, the chordae tendineae advantageously properly tether or position the valve leaflets such that the increase in pressure within the ventricle causes the valve to close, thereby preventing blood from leaking into the atrium and assuring that substantially all of the blood leaving the ventricle is ejected through the aortic valve 7 or pulmonic valve 9 and into the arteries of the body. Accordingly, proper function of the valves depends on a complex interplay between the annulus, leaflets, and sub-valvular apparatus. Lesions in any of these components can cause the valve to dysfunction and thereby lead to valve regurgitation.

Generally, there are three mechanisms by which a heart valve becomes regurgitant or incompetent; they include Carpentier's type I, type II and type III malfunctions. A Carpentier type I malfunction involves the dilation of the annulus such that normally functioning leaflets are distracted from each other and fail to form a tight seal (e.g., do not coapt properly). Included in a type I mechanism malfunction are perforations of the valve leaflets, as in endocarditis. A Carpentier's type II malfunction involves prolapse of one or both leaflets above the plane of coaptation. This is the most common cause of mitral regurgitation and is often caused by the stretching or rupturing of chordae tendineae normally connected to the leaflet. A Carpentier's type III malfunction involves restriction of the motion of one or more leaflets such that the leaflets are abnormally constrained below the level of the plane of the annulus. Leaflet restriction can be caused by rheumatic disease (IIIa) or dilation of the ventricle (IIIb).

One or more chambers in the heart 1 may be accessed in accordance with certain heart valve-repair procedures and/or other interventions. Access into a chamber in the heart may be made at any suitable site of entry. In some implementations, access is made to a chamber of the heart, such as a target ventricle (e.g., left ventricle) associated with a diseased heart valve, through the apical region 39. For example, access into the left ventricle 3 (e.g., to perform a mitral valve repair) may be gained by making a relatively small incision at the apical region 39, close to (or slightly skewed toward the left of) the median axis 27 of the heart. Access into the right ventricle 4 (e.g., to perform a tricuspid valve repair) may be gained by making a small incision into the apical region 39, close to or slightly skewed toward the right of the median axis 27 of the heart. Accordingly, the ventricle can be accessed directly via the apex, or via an off-apex location that is in the apical region 39 but slightly removed from the tip/apex, such as via lateral ventricular wall, a region between the apex and the base of a papillary muscle, or even directly at the base of a papillary muscle. In some implementations, the incision made to access the appropriate ventricle of the heart is no longer than about 1 mm to about 5 cm, from 2.5 mm to about 2.5 cm, or from about 5 mm to about 1 cm in length. When a percutaneous approach is sought, no incision into the apex region of the heart may be made, but rather access into the apical region 39 may be gained by direct needle puncture, for example by an 18-gauge needle, through which an appropriate repair instrument can be advanced.

Certain inventive features disclosed herein relate to certain heart valve repair systems and devices, and/or systems, process, and devices for repairing any other type of target organ tissue. In some implementations, a tissue anchor delivery device may be employed in repairing a mitral valve in patients suffering from degenerative mitral regurgitation or other condition. In some implementations, a transapical off-pump echo-guided repair procedure is implemented in which at least part (e.g., a shaft portion/assembly) of a valve repair system is inserted in the left ventricle and steered to the surface of the diseased portion of a target mitral valve leaflet and used to deploy/implant a tissue anchor 190 in the target leaflet. The tissue anchor 190 (e.g., sutureform formed into a bulky knot, T-fastener, grappling hook, spike, umbrella structure, pad) may advantageously be integrated or coupled with one or more artificial/synthetic cords serving a function similar to that of chordae tendineae. Such artificial cord(s) may comprise suture(s) and/or suture ends and/or tail portions associated with a knot-type tissue anchor 190 and may comprise any suitable or desirable material, such as expanded polytetrafluoroethylene (ePTFE) or the like. In some examples, cords and/or anchors described herein may be at least partially composed of organic, polymer, metal, and/or composite materials and/or may be at least partially ceramic-based.

Processes for repairing a target organ tissue, such as repair of mitral valve leaflets to address mitral valve regurgitation, can include inserting a tissue anchor delivery device, such as a delivery device as described in PCT Application No. PCT/US2012/043761, (published as WO 2013/003228, and referred to herein as “the '761 PCT Application”) and/or in PCT Application No. PCT/US2016/055170 (published as WO 2017/059426 and referred to herein as “the '170 PCT Application”), the entire disclosures of which are incorporated herein by reference, into a body and extending a distal end of the delivery device to a proximal side of the target tissue (e.g., leaflet).

The '761 PCT Application and the '170 PCT Application describe in detail methods and devices for performing non-invasive procedures to repair a cardiac valve, such as a mitral valve. Such procedures include procedures to repair regurgitation that occurs when the leaflets of the mitral valve do not coapt properly at peak contraction pressures, resulting in an undesired backflow of blood from the ventricle into the atrium. As described in the '761 PCT Application and the '170 PCT Application, after the malfunctioning cardiac valve has been assessed and the source of the malfunction verified, a corrective procedure can be performed. Various procedures can be performed in accordance with the methods described therein to effectuate a cardiac valve repair, which may depend on the specific abnormality and the tissues involved.

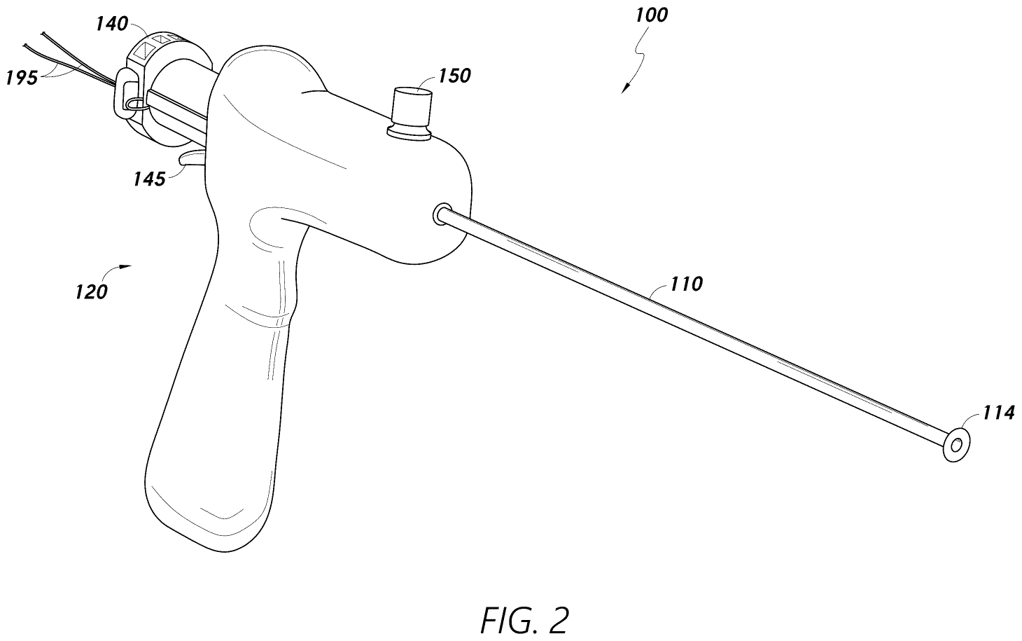

FIG. 2 is a perspective view of a tissue anchor delivery system 100 in accordance with one or more examples. The tissue anchor delivery system 100 may be used to repair a heart valve, such as a mitral valve, and improve functionality thereof. For example, the tissue anchor delivery system 100 may be used to reduce the degree of mitral regurgitation in patients suffering from mitral regurgitation caused by, for example, midsegment prolapse of valve leaflets as a result of degenerative mitral valve disease. In order to repair such a valve, the tissue anchor delivery system 100 may be utilized to deliver and anchor tissue anchors, such as suture-knot-type tissue anchors, in a prolapsed valve leaflet. As described in detail below, such procedures may be implemented on a beating heart.

The delivery system 100 includes a rigid elongate tube no forming at least one internal working lumen. The term “lumen” is used herein according to its broad and ordinary meaning, and may refer to a physical structure forming a cavity, void, pathway, or other channel, such as an at least partially rigid elongate tubular structure, or may refer to a cavity, void, pathway, or other channel, itself, that occupies a space within an elongate structure (e.g., a tubular structure). Therefore, with respect to an elongate tubular structure, such as a shaft, tube, or the like, the term “lumen” may refer to the elongate tubular structure and/or to the channel or space within the elongate tubular structure. Although described in certain examples and/or contexts as comprising a rigid elongate tube, it should be understood that tubes, shafts, lumens, conduits, and the like disclosed herein may be either rigid, at least partially rigid, flexible, and/or at least partially flexible. Therefore, any such component described herein, whether or not referred to as rigid herein should be interpreted as possibly being at least partially flexible. In accordance with the present disclosure, the rigid elongate tube 110 may be referred to as a “shaft” for simplicity. Implementation of a valve-repair procedure utilizing the delivery system 100 can be performed in conjunction with certain imaging technology designed to provide visibility of the shaft 110 of the delivery system 100 according to a certain imaging modality, such as echo imaging. Generally, when performing a valve-repair procedure utilizing the tissue anchor delivery system 100, the operating physician may advantageously work in concert with an imaging technician, who may coordinate with the physician to facilitate successful execution of the valve-repair procedure.

In addition to the delivery shaft 110, the delivery system 100 may include a plunger feature 140, which may be used or actuated to manually deploy a leaflet anchor, which may include a pre-formed knot, such as a bulky knot 190 as described in detail below. The tissue anchor delivery system 100 may further include a plunger lock mechanism 145, which may serve as a safety lock that locks the valve delivery system until ready for use or deployment of a leaflet anchor as described herein. The plunger 140 may have associated therewith a suture-release mechanism, which may be configured to lock in relative position one or more pairs of suture ends/tails 195 associated with a pre-formed knot anchor (not shown) to be deployed. For example, the suture portions 195 may be ePTFE sutures. The system 100 may further comprise a flush port 150, which may be used to de-air the lumen of the shaft 110. For example, heparinized saline flush, or the like, may be connected to the flush port 150 using a female Luer fitting to de-air the valve repair system 100.

The one or more lumens of the shaft 110 may be configured to house one or more needles (not shown) that may be configured to be wrapped at least in part with one or more pre-formed knot sutureform anchors 190, as described in detail herein. In some examples, the shaft 110 may be configured to form a relatively low profile. For example, the shaft 110 may have a diameter of approximately 0.5 inches less. The shaft 110 may be associated with an atraumatic tip 114 feature. The atraumatic tip 114 can be an echogenic leaflet-positioner component, which may be used for deployment and/or positioning of the suture-type tissue anchor. In some examples, the tip 114 may be cone-shaped and/or may have any other shape. The atraumatic tip 114, disposed at the distal end of the shaft 110, may be configured to have deployed therefrom one or more wrapped pre-formed suture knots 190 (e.g., sutureforms), as described herein.

The atraumatic tip 114 and/or distal portion of the shaft 110 may be referred to as an “end effector.” In some examples, the atraumatic tip 114 may have a generally conical shape and/or may be configured to form a generally circular platform and/or area of contact with the leaflet 154. In addition to one or more pre-formed knot sutureforms and/or associated needles, the shaft 110 may be configured to house one or more elongated knot pusher tubes (not shown; also referred to herein as “pushers”), which may be actuated simultaneously or sequentially using the plunger 140 in some examples. As described in further detail below, the tip 114 can provide a surface against which the target valve leaflet may be held in connection with deployment of a leaflet anchor.

The delivery system 100 may be used to deliver one or more tissue anchors, as described in greater detail below. For example, the delivery system 100 may be utilized to deliver one or more tissue anchors 190 (e.g., bulky knots) on a distal side of a mitral valve leaflet. While some examples described herein may refer to and/or describe “bulky knot” type tissue anchors 190, the delivery systems described herein may be configured for delivery of any of a variety of anchor types, including T-fasteners, hooks (e.g., grappling hooks), spikes, umbrella- and/or disc-shaped structures, and/or pads. The tip 114 (e.g., end effector), can be placed in contact with the ventricular side of a leaflet of a mitral valve. The tip 114 can be coupled to the distal end portion of the shaft 110, wherein the proximal end portion of the shaft 110 may be coupled to a handle portion 120 of the delivery system 100, as shown. Generally, the one or more elongate pushers (not shown) may be configured to be movably disposed within the one or more lumens of the shaft 110 and/or coupled to a pusher hub (not shown) that may be movably disposed within the handle 120 and/or releasably coupled to the plunger 140. One or more needles (not shown) carrying one or more pre-formed tissue anchor sutureforms can be movably disposed within lumens of the one or more pushers and/or coupled to a needle hub (not shown) that may be coupled to the plunger 140. The plunger 140 can be used to actuate or move the one or more needles and/or pushers during deployment of one or more distal anchors 190 and/or may be movably disposed at least partially within the handle 120. For example, the handle 120 may define one or more lumens in which the plunger 140 can be moved. During operation, each of the one or more pushers may also move within one or more lumens of the handle 120. The plunger lock 145 can be used to prevent the plunger 140 from moving within the handle 120 during storage and prior to performing a procedure to deploy one or more tissue anchors.

One or more needles may have a pre-formed knot 190 disposed about a distal portion thereof while maintained in the shaft 110. For example, a pre-formed knot 190 may be formed of one or more sutures configured in a coiled sutureform having a plurality of winds/turns around a needle over a portion of the needle that may be associated with a longitudinal slot in the needle that can run from the distal end thereof. Although the term “sutureform” is used herein, it should be understood that such components/forms may comprise suture, wire, and/or any other elongate material wrapped or formed in a desired configuration. The coiled sutureform can be provided or shipped disposed around the needle. In some examples, one or more suture tails 195 (e.g., two per needle) may extend from the coiled sutureform. The suture tails 195 may extend through the lumen of the needle and/or through a passageway of the plunger 140 and may exit the plunger 140 at a proximal end portion thereof. The coiled sutureform may advantageously be configured to be formed into a suture-type tissue anchor 190 (referred to herein as a “bulky knot”) in connection with an anchor-deployment procedure, as described in more detail below. The coiled sutureform can be configurable to a knot/deployed configuration by approximating opposite ends of the coiled portion thereof towards each other to form one or more loops.

The delivery device can further include a suture/tether catch mechanism (not shown) coupled to the plunger 140 at a proximal end of the delivery system 100, which may be configured to releasably hold or secure one or more sutures extending through the delivery system 100 during delivery of a tissue anchor as described herein. The suture catch can be used to hold the suture(s) with a friction fit and/or with a clamping force and/or can have a lock that can be released after the tissue anchor has been deployed/formed into a bulky knot, as described herein.

The anchor delivery system 100 can be used in beating heart mitral valve repair procedures. In some examples, the shaft 110 of the delivery system 100 can be configured to extend and/or contract with the beating of the heart. During systolic contraction, the median axis of the heart generally shortens. For example, the distance from the apex 19 of the heart to the valve leaflets 52, 54 can vary by about 1 centimeter (cm) to about 2 cms with each heartbeat in some patients. In some examples, the length of the shaft 110 that protrudes from the handle 120 can change with the length of the median axis of the heart. That is, distal end of the shaft 110 can be configured to be floating such that the shaft can extend and retract with the beat of the heart so as to maintain contact with the target mitral valve leaflet.

Advancement of the delivery system 100 may be performed in conjunction with echo imaging, direct visualization (e.g., direct transblood visualization), and/or any other suitable remote visualization technique/modality. With respect to cardiac procedures, for example, the delivery system 100 may be advanced in conjunction with transesophageal (TEE) guidance and/or intracardiac echocardiography (ICE) guidance to facilitate and to direct the movement and proper positioning of the device for contacting the appropriate target cardiac region and/or target cardiac tissue (e.g., a valve leaflet, a valve annulus, or any other suitable cardiac tissue). Typical procedures that can be implemented using echo guidance are set forth in Suematsu, Y., J. Thorac. Cardiovasc. Surg. 2005; 130:1348-56 (“Suematsu”), the entire disclosure of which is incorporated herein by reference.

According to some implementations of valve-repair procedures, an incision into the apical region 19 of the appropriate ventricle 3 of the heart is made. For example, an introducer port device containing one or more fluid-retention valves to prevent blood loss and/or air entry into the ventricle 3, may be inserted into the site of entry. Once inside the chamber (e.g., ventricle 3), the shaft 110 of the delivery system 100 may be advanced through the lumen of the introducer. In some examples, a sheath may be inserted through the introducer, through which one or more other instruments are advanced. For example, an endoscope may first be advanced into the chamber (e.g., ventricle 3) to visualize the ventricle 3, the valve 6, and/or the sub-valvular apparatus. By use of an appropriate endoscope, a careful analysis of the malfunctioning valve 6 may be performed. Each segment of each leaflet may be carefully assessed to determine its pliability, integrity, and motion. Based on this assessment, the practitioner can determine whether the valve can indeed be repaired or must be replaced. The motion of the leaflets 52, 154 can be classified as slightly dysfunctional, prolapsed, or restricted and based on this classification, the necessary steps of the repair can be determined.

Mitral valve regurgitation generally increases the workload on the heart and may lead to very serious conditions if left untreated, such as decreased ventricular function, pulmonary hypertension, congestive heart failure, permanent heart damage, cardiac arrest, and ultimately death. Since the left heart is primarily responsible for circulating the flow of blood throughout the body, malfunction of the mitral valve 36 is particularly problematic and often life threatening. Methods and devices are provided herein, as well as in the '761 PCT Application and the '170 PCT Application, for performing non-invasive procedures to repair a cardiac valve, such as a mitral valve. Such procedures include procedures to repair regurgitation that occurs when the leaflets of the mitral valve do not coapt properly at peak contraction pressures, resulting in an undesired backflow of blood from the ventricle into the atrium. As described in the '761 PCT Application and the '170 PCT Application, after the malfunctioning cardiac valve has been assessed and the source of the malfunction verified, a corrective procedure can be performed. Various procedures can be performed in accordance with the methods described therein to effectuate a cardiac valve repair, which will depend on the specific abnormality and the tissues involved.

After a minimally invasive approach is determined to be advisable, one or more incisions may be made proximate to the thoracic cavity to provide a surgical field of access. The total number and length of the incisions to be made depend on the number and types of the instruments to be used as well as the procedure(s) to be performed. The incision(s) may advantageously be made in such a manner as to be minimally invasive. As referred to herein, the term “minimally invasive” means in a manner by which an interior organ or tissue may be accessed with relatively little damage being done to the anatomical structure through which entry is sought. For example, a minimally invasive procedure may involve accessing a body cavity by a small incision of, for example, approximately 5 cm or less made in the skin of the body. The incision may be vertical, horizontal, or slightly curved. If the incision is located along one or more ribs, it may advantageously follow the outline of the rib. The opening may advantageously extend deep enough to allow access to the thoracic cavity between the ribs or under the sternum and is preferably set close to the rib cage and/or diaphragm, dependent on the entry point chosen.

In one example method, the heart may be accessed through one or more openings made by one or more small incision in a portion of the body proximal to the thoracic cavity, such as between one or more of the ribs of the rib cage of a patient, proximate to the xyphoid appendage, or via the abdomen and diaphragm. Access to the thoracic cavity may be sought to allow the insertion and use of one or more thorascopic instruments, while access to the abdomen may be sought to allow the insertion and use of one or more laparoscopic instruments. Insertion of one or more visualizing instruments may then be followed by transdiaphragmatic access to the heart. Additionally, access to the heart may be gained by direct puncture (e.g., via an appropriately sized needle, for example an 18-gauge needle) of the heart from the xyphoid region. Accordingly, the one or more incisions should be made in such a manner as to provide an appropriate surgical field and access site to the heart in the least invasive manner possible. Access may also be achieved using percutaneous methods, further reducing the invasiveness of the procedure. See, e.g., “Full-Spectrum Cardiac Surgery Through a Minimal Incision Mini-Sternotomy (Lower Half) Technique,” Doty et al., Annals of Thoracic Surgery 1998; 65(2): 573-77 and “Transxiphoid Approach Without Median Sternotomy for the Repair of Atrial Septal Defects,” Barbero-Marcial et al., Annals of Thoracic Surgery 1998; 65(3): 771-74, the entire disclosures of each of which are incorporated herein by reference.

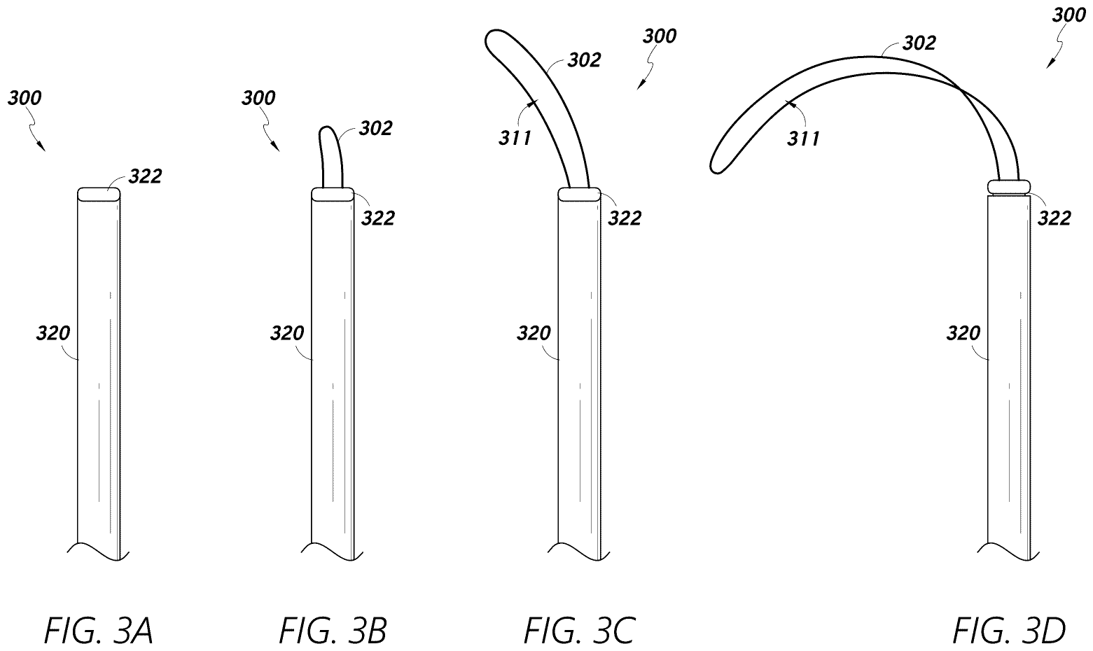

FIGS. 3A-3D illustrate a delivery system 300 for delivering an example support device 302 via a shaft 320 in accordance with one or more examples. The shaft 320 (e.g., a catheter) may have a generally tubular form and/or may comprise an inner lumen configured to receive the support device 302. FIG. 3A illustrates an initial stage in which the support device 302 is entirely situated within the shaft 320. In FIG. 3B, a distal portion of the support device 302 may be extended beyond a distal member 322 of the shaft 320. In some examples, the support device 302 may be configured to assume an at least partially compressed and/or collapsed configuration while within the shaft 320. For example, the device 302 may comprise a distal portion forming a loop and/or lasso about a space 311 between an outer frame of the device 302. The space 311 may be compressed and/or the lines of the device 302 may be pressed together while the device 302 is within the shaft 320. Moreover, the device 302 may assume compressed and/or collapsed shape while the device 302 is within the shaft 320. For example, the device 302 may be shape-set to have a curved, hook-shaped, and/or semi-circular form, as shown in FIG. 3D. While the device 302 is situated within the shaft 320, the device 302 may be configured to straighten in response to force applied by the walls of the shaft 320. As the device 302 exits the shaft 320, the device 302 may be configured to return and/or naturally assume the curved form shown in FIG. 3D.

While the support device 302 is shown in FIGS. 3A-3D having a loop and/or lasso form, the support device 302 may have other forms. For example, the device 302 may comprise a single line and/or length of material. In some examples, the device 302 may comprise multiple lines which may be interconnected (e.g., braided) and/or may be at least partially flexible to adjust when placed within the shaft 320 and/or may be at least partially rigid to support a valve leaflet. The support device 302 may be configured to be retracted into the support shaft 320 following delivery of one or more sutures.

The support device 302 (e.g., support arm and/or platform) may be configured to naturally assume the concave and/or convex form shown in FIG. 3D. The device 302 may be configured to approximate a curvature of one or more valve leaflets. In some examples, the device 302 may assume a generally semi-circular shape.

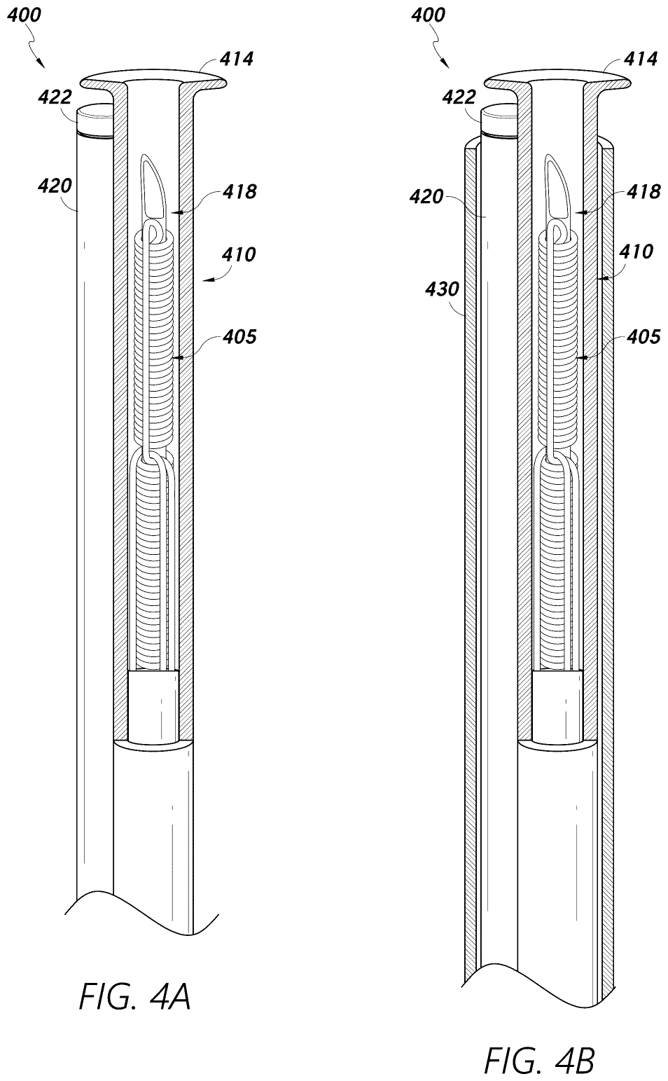

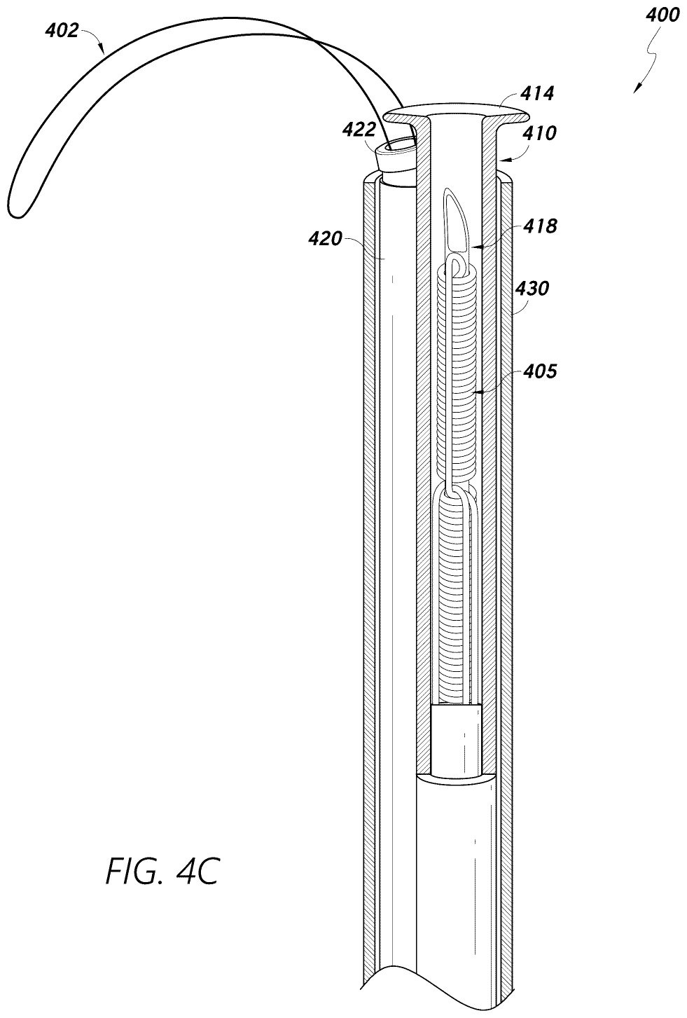

FIGS. 4A-4C illustrate a cutaway view of a delivery system 400 for delivering one or more sutures 405 (e.g., suture anchors and/or loops) and/or support devices 402 at or near one or more valve leaflets of a heart in accordance with one or more examples. A suture 405 may have any suitable form and/or may be configured to assume a desired form to establish a secure attachment with one or more leaflets. For example, a suture 405 may comprise a coiled sutureform configured to form a bulky knot when removed from a needle 418. In some examples, a suture 405 may be configured to form a loop and/or may be configured to loop through a leaflet.

The delivery system 400 may comprise one or more devices and/or components which may be delivered via an outer shaft 430 (e.g., a catheter). In some examples, one or more tissue anchors 405 may be delivered via an inner shaft 410 (e.g., a catheter). The tissue anchors 405 may comprise one or more sutures forming one or more coils, as shown in FIGS. 4A-4C. In some examples, the one or more tissue anchors 405 may be wrapped at least partially around a needle 418 and/or shaft. The needle 418 may comprise a pointed tip at a distal end of the needle 418 and/or may comprise other features for facilitating delivery of the one or more sutures 405 through one or more valve leaflets.

In some examples, more than one suture 405 may be simultaneously delivered into a body and/or to a leaflet via the needle 418 and/or inner shaft 410. For example, multiple anchors 405 may be situated end-to-end along a surface of the needle 418, as shown in FIGS. 4A-4C.

The delivery system 400 may further comprise a support shaft 420 configured for delivery of one or more support devices 402. In some examples, the support shaft 420 may comprise an inner lumen configured to receive one or more support devices 402. The support shaft 420 may be configured to be situated approximately adjacent to and/or parallel with the inner shaft 410 and/or the outer shaft 430.

In some examples, the support shaft 420 may comprise a distal member 422, which may form a distal end of the shaft 420. The distal member 422 may be configured to move with respect to other portions of the support shaft 420. For example, the distal member 422 may comprise a pivot and/or similar mechanism configured to adjust an angle and//or facing direction of the distal end of the of support shaft 420. The distal member 422 may be configured to pivot to cause the support device 402 to exit the support shaft 420 at an angle (e.g., at an approximately 45-degree angle). For example, the distal member 422 may be configured to allow the support device 402 to exit the support shaft 420 away from the inner shaft to provide space for the support device 402 to exit the support shaft 420 and/or expand.

In some examples, the inner shaft 410 may comprise an end effector 414 at a distal end of the inner shaft 410. The end effector 414 may have an increased diameter with respect to other portions of the inner shaft 410 and/or may be configured to tent and/or otherwise contact an underside and/or upper side of a valve leaflet. The end effector 414 may have a conical form in which the diameter of the end effector 414 may increase as the end effector 414 extends to a distal end of the inner shaft 410.

The support arm 402 may have a textured surface to improve grip and/or prevent slippage. For example, a surface of the support arm 402 can comprise a series of sharp and/or semi-sharp teeth in a consistent and/or inconsistent alignment along the support arm 402. In some examples, the support arm 402 may have a variable and/or irregular contact profile (e.g., irregularly sized and/or spaced teeth).

A distal portion of the support device/arm 402 may be configured to extend generally perpendicularly from the support shaft 420 and/or a proximal portion (e.g., a portion of the support arm 402 within the support shaft 420) of the support arm 402.

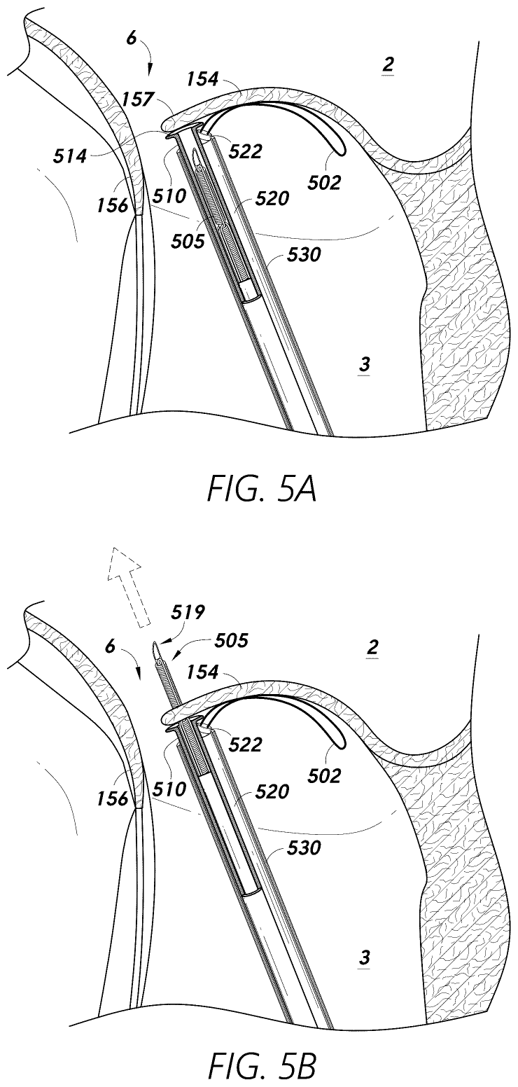

FIGS. 5A and 5B illustrate delivery of one or more suture anchors via an example delivery system through one or more leaflets 154 of a heart valve 6 in accordance with one or more examples. As shown in FIGS. 5A and 5B, the delivery system may comprise a support device 502 (e.g., an arm and//or platform) configured to extend at least partially along an underside and/or upper side of the leaflet 154. The support device 502 may be configured to facilitate delivery of one or more suture anchors at or near an edge portion 157 of the leaflet 154 (e.g., near a second leaflet 156). The support device 502 may be configured to naturally assume a curved form (e.g., a convex form) to approximate a curvature of the leaflet 154.

The support device 502 may have any suitable and/or structure. In some examples, the support device 502 may comprise (e.g., at a distal end of the support device 502) a loop and/or lasso formed from cords and/or other lines. However, other support arm 502 designs may be used, including a platform at least partially composed of a wireframe and/or a webbing and/or sheet. In some examples, the support arm 502 may comprise, at the distal end, an increased size and/or surface area to maximize an area of contact between the support arm 502 and the leaflet 154. For example, the support arm 502 may increase in width and/or surface area towards the distal end of the support arm 502. In some examples, at least a portion of the support arm 502 may comprise ridges, grooves, protrusions, and/or other features to increase a texture of the support arm 502 and/or to increase friction between the support arm 502 and the leaflet 154. For example, the support arm 502 may be laser-cut to add divots to the support arm 502. At least a portion of the support arm 502 may comprise a coating and/or covering to additionally and/or alternatively increase friction. For example, cords forming the support arm 502 may be coated and/or covered with various materials. While the support arm 502 is shown comprising cords forming a looped end with an opening through the looped end, the support arm 502 may comprise a covering configured to extend at least partially across the opening formed by the cords.

In some examples, the support device 502 may be configured to extend from a support shaft 520 of the delivery system. The support shaft 520 may comprise a distal member 522 configured to facilitate delivery of the support device 502 away from an inner shaft 510 of the delivery system and/or along the underside of the leaflet 154. The inner shaft 510 and/or support shaft 520 may be delivered via an outer shaft 530.

As shown in FIG. 5B, the one or more suture anchors 505 and/or sutures may be situated along a needle 519 configured to drive through the leaflet 154. With the support device 502 supporting the leaflet 154, the needle 519 may be enabled to more effectively pass through the leaflet 154 at the distal edge portion 157 of the leaflet 154.

The target site of the valve 6 may be slowly approached from the ventricle side thereof by advancing the distal end of the shaft 510 along or near to the posterior wall of the ventricle 3 (e.g., left ventricle) without contacting the ventricle wall. Successful targeting and contacting of the target location on the leaflet 154 can depend at least in part on accurate visualization of the shaft 510 and/or tip/end effector 514 throughout the process of advancing the tip 514 to the target site. Generally, echocardiographic equipment may be used to provide the necessary or desired intra-operative visualization of the shaft 510 and/or tip 514.

Once the tip 514 is positioned in the desired position, the distal end of the shaft 510 and the tip 514 may be used to drape, or “tent,” the leaflet 154 to better secure the tip 514 in the desired position, as shown in FIG. 5A. Draping/tenting may advantageously facilitate contact of the tip 514 with the leaflet 154 throughout one or more cardiac cycles, to thereby provide more secure or proper deployment of leaflet anchor(s). The target location may advantageously be located relatively close to the free edge 157 of the target leaflet 154 to minimize the likelihood of undesirable intra-atrial wall deployment of the anchor. Navigation of the tip 514 to the desired location on the underside of the target valve leaflet 154 may be assisted using echo imaging, as described in detail herein. Echo imaging may be relied upon to confirm correct positioning of the tip 514 prior to anchor/knot deployment.

The inner shaft 510 can be placed over the anterior commissure to provide a suitable attack position for deployment of the first anchor 505. Verification of the first anchoring location can be obtained using imaging. In some implementations, the first anchor 505 is placed close to the leaflet hinge, as anterior as possible in the annulus, close to the anterior commissure.

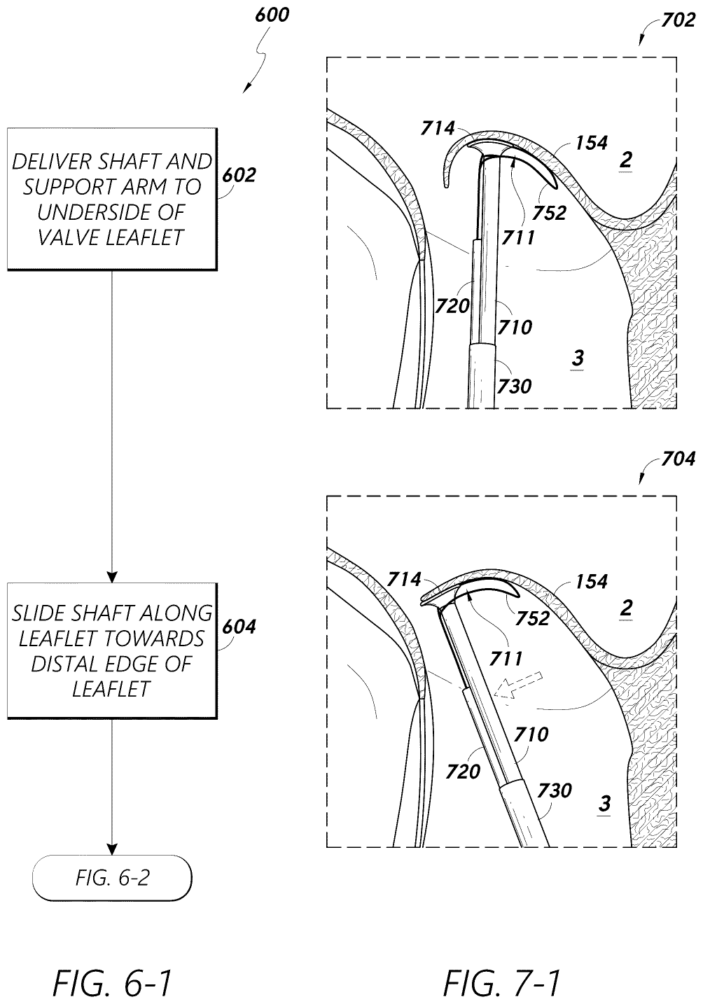

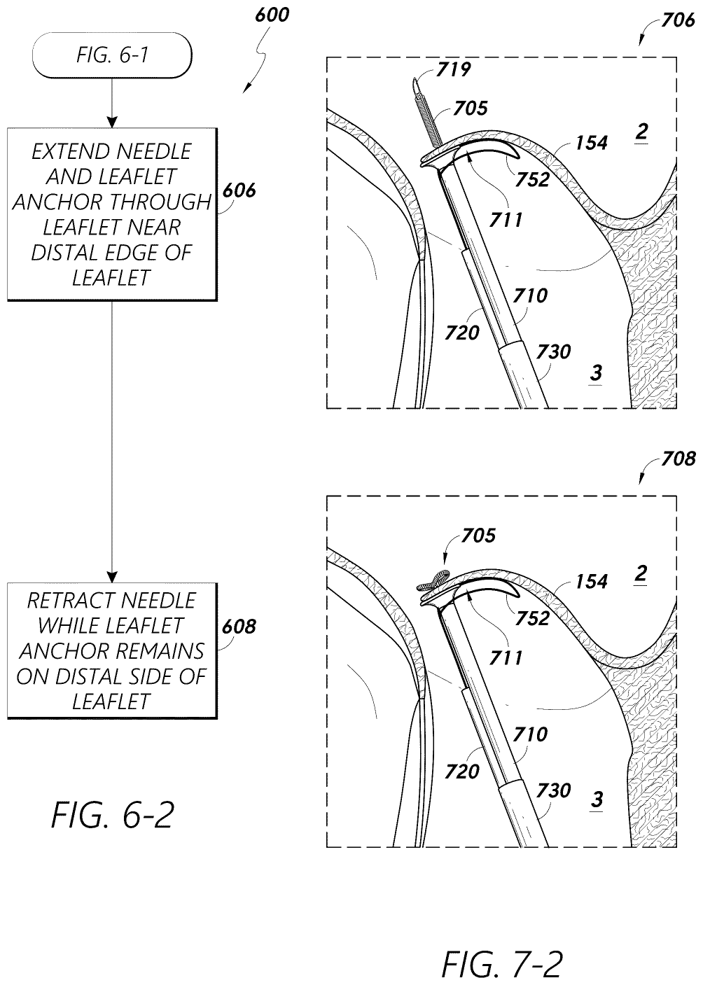

FIG. 6 (FIGS. 6-1 and 6-2) is a flow diagram illustrating a process 600 for delivering one or more leaflet anchors to a valve leaflet in accordance with one or more examples of the present disclosure. FIG. 7 (FIGS. 7-1 and 7-2) provides illustrations corresponding to steps of the process 600 described in FIG. 6.

At step 602, the process 600 involves delivering one or more shafts (e.g., an outer shaft 730, an inner shaft 710, and/or a support shaft 720) and/or a support arm 752 to an underside of a valve leaflet 154, as shown in image 702 of FIG. 7. While the support arm 752 is shown extending along an underside of the leaflet 154, the devices and/or processes described herein may be additionally or alternatively applied to transapical and/or transcatheter procedures involving delivery to an upper side of the leaflet 154. In some examples, one or more shafts (e.g., the inner shaft 710) may comprise an end effector 714 configured to contact and/or tent a portion of the underside and/or upper side of the leaflet 154. The support arm 752 can extend from the support shaft 720. In some examples, the one or more shafts may initially be delivered such that the end effector 714 contacts the leaflet 154 at a point that is relatively distal from an edge portion of the leaflet 154.

In some examples, the support arm 752 may comprise a looped cord forming a space 711 between lines of the support arm 752. As shown in FIG. 7, the support arm 752 can be at least partially looped around the inner shaft 710 before and/or during deployment of the support arm 752 and/or suture anchors. However, the support arm 752 may not loop around the inner shaft 710 in other examples.

At step 604, the process 600 involves sliding the one or more shafts (e.g., sliding the end effector 714) along the underside of the leaflet 154 until the end effector 714 is situated at least partially below an edge portion of the leaflet 154, as shown in image 704 of FIG. 7. The support arm 752 may similarly slide along the leaflet 154 and/or may extend along the leaflet 154 to provide support to the leaflet 154.

At step 606, the process 600 involves extending a needle 719 carrying one or more suture anchors 705 at least partially through the leaflet 154 at the edge portion of the leaflet 154, as shown in image 706 of FIG. 7. The end effector 714 and/or support arm 752 may remain in contact with the underside of the leaflet 154 while the needle 719 passes through the leaflet 154. In some examples, the needle 719 may comprise a pointed tip and/or may be configured to puncture through the leaflet 154.

At step 608, the process 600 involves retracting the needle 719 while at least one suture anchor 705 remains situated at a distal side of the leaflet 154, as shown in image 708 of FIG. 7. In some examples, the suture anchor 705 may be configured to form a knot (e.g., a bulky knot) in response to retraction of the needle 719.

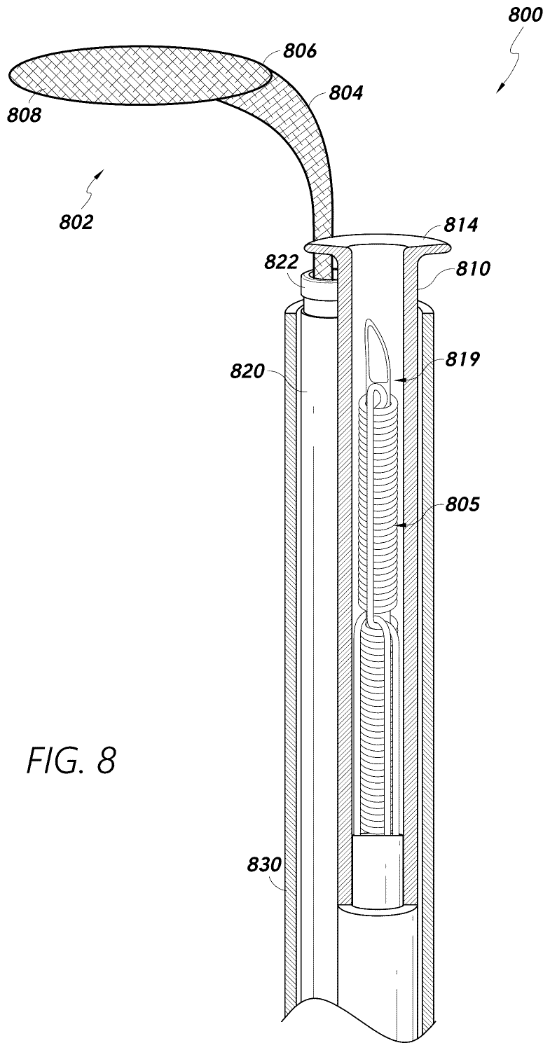

FIG. 8 illustrates a cutaway view of a delivery system 800 for delivering one or more suture anchors 805 and/or support devices 802 at or near one or more valve leaflets of a heart in accordance with one or more examples. The delivery system 800 may comprise one or more devices and/or components which may be delivered via an outer shaft 830 (e.g., a catheter). In some examples, one or more tissue anchors 805 may be delivered via an inner shaft 810 (e.g., a catheter). The tissue anchors 805 may comprise one or more sutures forming one or more coils, as shown in FIG. 8. In some examples, the one or more tissue anchors 805 may be wrapped at least partially around a needle 819 and/or shaft. The needle 819 may comprise a pointed tip at a distal end of the needle 819 and/or may comprise other features for facilitating delivery of the one or more suture anchors 805 through one or more valve leaflets.

In some examples, more than one suture anchor 805 may be simultaneously delivered into a body and/or to a leaflet via the needle 819 and/or inner shaft 810. For example, multiple anchors 805 may be situated end-to-end along a surface of the needle 819, as shown in FIG. 8.

The delivery system 800 may further comprise a support shaft 820 configured for delivery of one or more support devices 802. In some examples, the support shaft 820 may comprise an inner lumen configured to receive one or more support devices 802. The support shaft 820 may be configured to be situated approximately adjacent to and/or parallel with the inner shaft 810 and/or the outer shaft 830.

In some examples, the support shaft 820 may comprise a distal member 822, which may form a distal end of the shaft 820. The distal member 822 may be configured to move with respect to other portions of the support shaft 820. For example, the distal member 822 may comprise a pivot and/or similar mechanism configured to adjust an angle and//or facing direction of the distal end of the of support shaft 820. The distal member 822 may be configured to pivot to cause the support device 802 to exit the support shaft 820 at an angle (e.g., at an approximately 45-degree angle). For example, the distal member 822 may be configured to allow the support device 802 to exit the support shaft 820 away from the inner shaft to provide space for the support device 802 to exit the support shaft 820 and/or expand.

In some examples, the inner shaft 810 may comprise an end effector 814 at a distal end of the inner shaft 810. The end effector 814 may have an increased diameter with respect to other portions of the inner shaft 810 and/or may be configured to tent and/or otherwise contact an underside of a valve leaflet. The end effector 814 may have a conical form in which the diameter of the end effector 814 may increase as the end effector 814 extends to a distal end of the inner shaft 810.

As shown in FIG. 8, the support device 802 can comprise various components, which can include one or more arms 804 and/or a platform 806. The platform 806 may comprise a distal portion of the support device 802 (e.g., arm) and/or the arms 804 may comprise a proximal portion of the support device 802. The one or more arms 804 can have a generally curved form to hold the platform at an approximately 90-degree angle with respect to a longitudinal and/or central axis of the support shaft 820 and/or outer shaft 830. In some examples, the one or more arms 804 may be configured to assume a generally straight and/or linear configuration while within the support shaft 820 and/or may be configured to naturally bend and/or expand upon removal from the support shaft 820. In some examples, the one or more arms 804 may be interconnected by a webbing 808 and/or sheet configured to extend at least partially between the arms 804. The webbing 808 may be composed of any suitable material (e.g., fabric, rubber, polyester and/or may comprise a mesh and/or network of one or more cords and/or threads). The webbing covering the platform 806 may have a generally circular shape. A distal portion of the support device 802 (e.g., the platform 806 and/or webbing 808 within the platform 806) may be configured to extend generally perpendicularly (e.g., at approximately 90-degrees) from the support shaft 820 and/or proximal portion (e.g., a portion within the support shaft 820) of the support device 802.

In some examples, the platform 806 may have a generally curved shape, similar to the support device 402 of FIG. 4. For example, the platform 806 may have a generally convex and/or concave form. The webbing 808 at the platform 806 may similarly have a curved form.

The platform 806 may extend from the one or more arms 804 and/or may comprise an extension from the one or more arms 804. While the platform 806 is shown having a generally circular form, the platform 806 may have any suitable shape and/or size. The platform 806 may comprise a webbing 808 to extend between wires and/or cords of the platform 806. The platform 806 may be configured to be situated at least partially below a leaflet and/or may be configured to contact and/or extend along an underside of the leaflet.

The support device 802 may be at least partially compressible and/or may be configured to compress while within the support shaft 820. For example, the webbing 808, platform 806, and/or arms 804 may be configured to fold and/or bend to assume a smaller profile to fit within the support shaft 820 and/or may be configured to expand and/or unfurl naturally in response to removal from the support shaft 820. The webbing 808 can comprise a textured and/or expandable (e.g., stretchy) surface configured to increase grip and/or reduce risk of slipping and/or to cause expansion of the webbing 808. The platform 806 can comprise a generally rigid and/or semi-rigid outer rim.

The one or more arms 804 may assume an arched and/or bent shape in response to removal from the support shaft 820. For example, the arms 804 may bend to an approximately 90-degree angle with respect to the support shaft 820 and/or with respect to a portion of the arms 804 within the support shaft 820. In some examples, a separation and/or distance between the one or more arms 804 may increase towards the platform 806, as shown in FIG. 8.

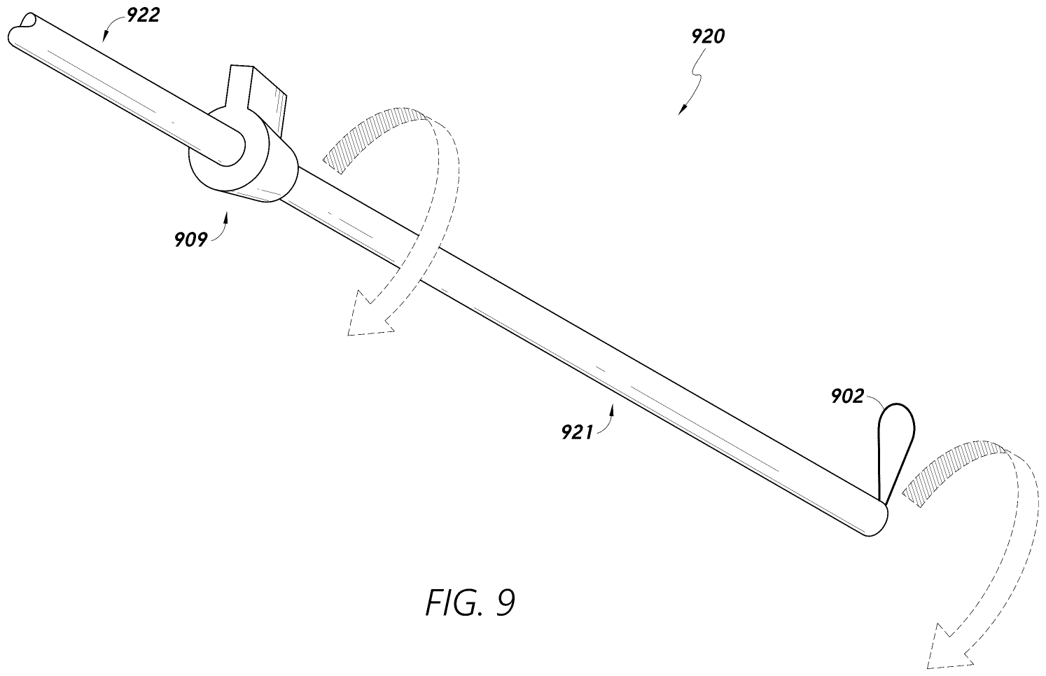

FIG. 9 illustrates an example support shaft 920 for delivering one or more support devices 902 in accordance with one or more examples. In some examples, the support shaft 920 may comprise a rotation device 909 configured to rotate at least a portion of the support shaft 920 to adjust a deployment angle and/or direction of the support device 902. For example, rotation of the support shaft 920 position the support arm 902 below and/or above a leaflet. The support shaft 920 may comprise multiple separate and/or interconnected components, which can include a proximal portion 922 and/or a distal portion 921. The rotation device 909 may be configured to cause rotation of the entire support shaft 920 and/or of only the distal portion 921 of the support shaft 920.

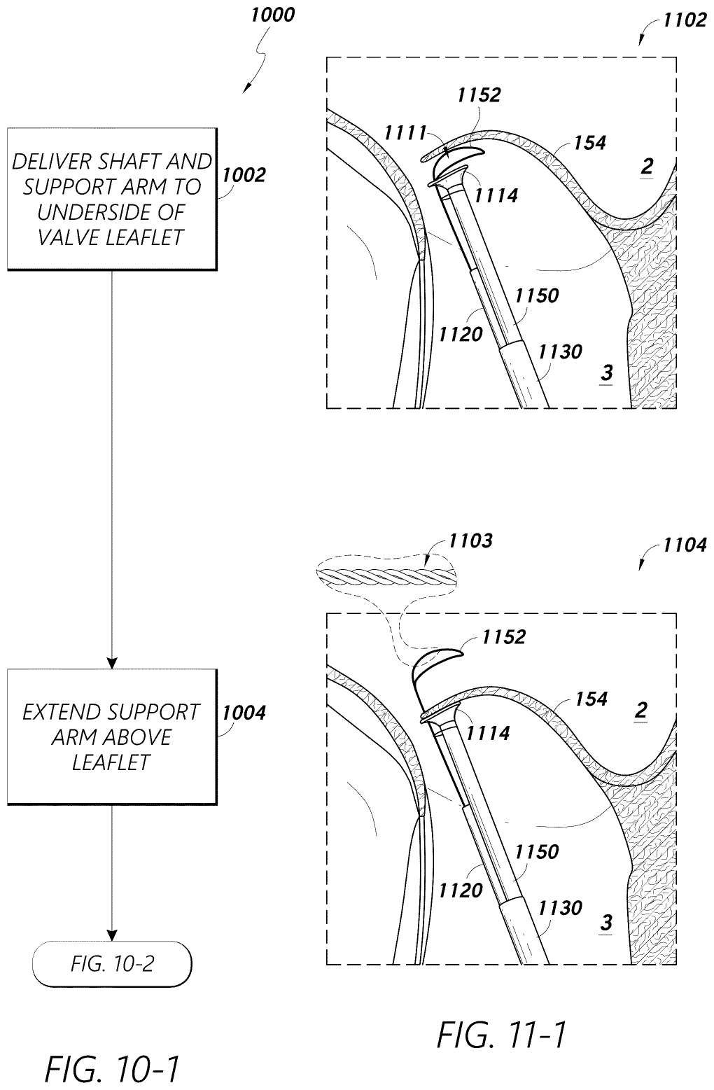

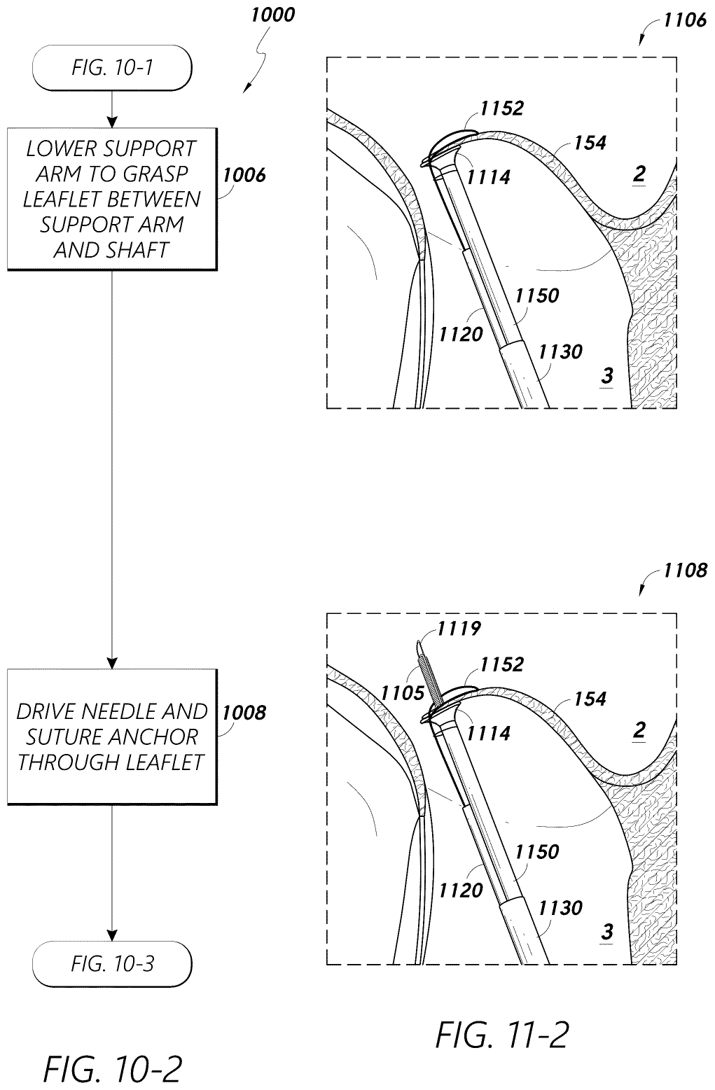

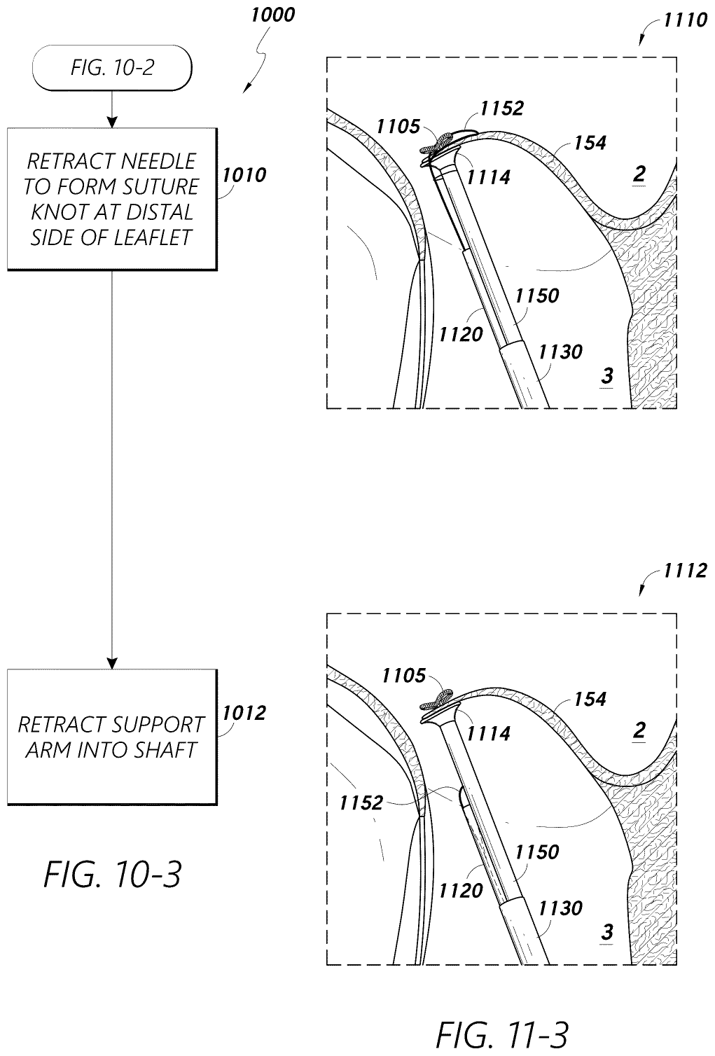

FIG. 10 (FIGS. 10-1, 10-2, and 10-3) is a flow diagram illustrating a process 1000 for delivering one or more leaflet anchors to a valve leaflet in accordance with one or more examples of the present disclosure. FIG. 11 (FIGS. 11-1, 11-2, and 11-3) provides illustrations corresponding to steps of the process 1000 described in FIG. 10.

At step 1002, the process 1000 involves delivering one or more shafts (e.g., an outer shaft 1130, an inner shaft 1150, and/or a support shaft 1120) and/or a support arm 1152 to an underside of a valve leaflet 154, as shown in image 1102 of FIG. 11. In some examples, one or more shafts (e.g., the inner shaft 1150) may comprise an end effector 1114 configured to contact and/or tent a portion of the underside of the leaflet 154. The support arm 1152 can extend from the support shaft 1120. In some examples, the one or more shafts may initially be delivered such that the end effector 1114 contacts the leaflet 154 at a point that is relatively distal from an edge portion of the leaflet 154.

In some examples, the support arm 1152 may comprise a looped cord forming a space 11n1 between lines of the support arm 1152. While the looped cord of the support arm 1152 is shown above the end effector 1114 in image 1102, at least a portion of the support arm 1152 (e.g., at least a portion of the looped cord) can remain within the support shaft 1120 during contact and/or tenting of the underside of the leaflet 154 by the end effector 1114. The support arm 1152 may extend at least partially over the end effector 1114 and/or may extend to the side of the end effector 1114. The end effector 1114 may be delivered to an underside of an edge portion of the leaflet 154 and/or may be slid to the edge portion of the leaflet 154 following initial contact between the end effector 1114 and the leaflet 154.

At step 1004, the process 1000 involves extending at least a portion of the support arm 1152 (e.g., a coiled loop, lasso, and/or platform of the support arm 1152) above the leaflet 154, as shown in image 1104 of FIG. 11. At least a portion of the support arm 1152 may be configured to extend at least partially over the leaflet 154.

As shown in image 1104, at least a portion of the support arm 1152 may comprise interwoven and/or braided lines 1103 and/or wire forms, which can include cords, threads, wires, and/or other similar devices. The support arm 1152 may be at least partially flexible and/or may have sufficient rigidity to provide support to the leaflet 154.

At step 1006, the process 1000 involves lowering the support arm 1152 to grasp and/or immobilize at least a portion of the leaflet 154 between at least a portion of the support arm 1152 (e.g., a platform and/or loop of the support arm 1152) and the end effector 1114, as shown in image 1106 of FIG. 11. In some examples, the portion of the support arm 1152 above the leaflet 154 may have a looped form forming an opening situated at least partially above the inner shaft 1150. The support arm 1152 may be configured to press down and/or downward on the leaflet 154 while the end effector 1114 and/or a second support arm below the leaflet 154 presses up and/or upward against the leaflet to grasp the leaflet 154.

At step 1008, the process 1000 involves extending a needle 1119 carrying one or more suture anchors 1105 at least partially through the leaflet 154 at the edge portion of the leaflet 154, as shown in image 1108 of FIG. 11. The end effector 1114 may remain in contact with the underside of the leaflet 154 and/or the support arm 1152 may remain in contact with an upper side of the leaflet 154 while the needle 1119 passes through the leaflet 154. In some examples, the needle 1119 may comprise a pointed tip and/or may be configured to puncture through the leaflet 154. The support arm 1152 and/or a looped portion of the support arm 1152 may be positioned such that the needle 1119 extends through an opening formed by the looped portion of the support arm 1152.

At step 1010, the process 1000 involves retracting the needle 1119 while at least one suture anchor 1105 remains situated at a distal side of the leaflet 154, as shown in image 1110 of FIG. 11. In some examples, the suture anchor 1105 may be configured to form a knot (e.g., a bulky knot) in response to retraction of the needle 1119.

At step 1012, the process 1000 involves retracting the support arm 1152 into the support shaft 1120 after the knot is formed, as shown in image 1112 of FIG. 11. In some examples, the support arm may be pressed and/or pulled up and/or over the knot prior to retracting the support arm 1152. The support arm 1152 may be configured to at least partially compress in response to being retracted into the support shaft 1120.

In some examples, multiple support arms 1152 may be used in a delivery process. For example, the support arm 1152 illustrated in FIG. 11 may be a first support arm 1152 configured to extend at least partially along an upper side of the leaflet 154. In addition, a second support arm (e.g., the support arm 752 of FIG. 7) may be extended along an underside of the leaflet 154 simultaneously with the first support arm 1152 and/or the first support arm 1152 and the second support arm may be simultaneously positioned on either side of the leaflet 154 to sandwich and/or grasp at least a portion of the leaflet 154 between the first support arm 1152 and the second support arm. The first support arm 1152 and optional second support arm may be delivered via the same support shaft 1120 and/or may be delivered via separate support shafts 1120.

In accordance with some implementations of the present disclosure, a delivery system comprises a first delivery shaft configured to deliver one or more sutures to a leaflet of a heart. The first delivery shaft has an end effector at a distal end of the first delivery shaft and configured to contact the leaflet. The delivery system further comprises a needle configured to extend from the distal end of the first delivery shaft and through the leaflet and a first support arm configured to contact and extend at least partially along the leaflet during delivery of the needle through the leaflet.

In some examples, each of the one or more sutures is wrapped at least partially around a distal portion of the needle and is configured to form a knot anchor when removed from the needle. The first support arm may be configured to extend at least partially along an underside of the leaflet.

The delivery system may further comprise a second support arm configured to extend at least partially along an upper side of the leaflet. In some examples, the first support arm is configured to extend at least partially along an upper side of the leaflet.

In some examples, the delivery system further comprises a second support arm configured to extend at least partially along an underside of the leaflet. The first support arm may be configured to press down on the leaflet while the second support arm presses up on the leaflet.

The first support arm may be configured to press down on the leaflet while the end effector presses up on the leaflet. In some examples, the delivery system further comprises a support shaft configured to deliver the first support arm.

In some examples, the end effector is configured to contact an underside of the leaflet. The end effector may be configured to contact an upper side of the leaflet.

The one or more sutures may be configured to form loops extending at least partially through the leaflet. In some examples, the first support arm forms a loop with an opening.

In some examples, the needle is configured to extend through the opening. The first support arm may comprise a sheet supported by a wireframe. A distal portion of the sheet may have a circular shape.

A distal portion of the first support arm may be configured to extend generally perpendicularly relative to a proximal portion of the first support arm. In some examples, the distal portion is configured to naturally extend generally perpendicularly in response to removal from a support shaft.

In some examples, the first support arm has a braided wire form. The first delivery shaft may comprise a distal portion configured to rotate independently of a proximal portion of the first delivery shaft.

Some implementations of the present disclosure relate to a method comprising delivering a first delivery shaft to an underside of a leaflet of a heart, the first delivery shaft having an end effector at a distal end of the first delivery shaft. The first delivery shaft carries a needle and one or more sutures. The method further comprises extending a first support arm along the leaflet, sliding the end effector towards an edge portion of the leaflet, and extending the needle through the leaflet at the edge portion while the first support arm and the end effector are in contact with the leaflet.

Each of the one or more sutures may be wrapped at least partially around a distal portion of the needle and is configured to form a knot anchor when removed from the needle. In some examples, the method further comprises extending the first support arm at least partially along an underside of the leaflet.

In some examples, the method further comprises extending a second support arm at least partially along an upper side of the leaflet. The method may further comprise delivering a support shaft carrying the first support arm to the underside of the leaflet.

The method may further comprise extending the first support arm at least partially above the leaflet. In some examples, the method further comprises lowering the first support arm to contact an upper side of the leaflet.

In some examples, the needle is extended through the leaflet while the leaflet is at least partially immobilized between the first support arm and the end effector. The needle may be extended through an opening of the first support arm above the leaflet.

The method may further comprise pressing the first support arm downward against the leaflet while pressing the end effector upward against the leaflet. In some examples, the method further comprises extending a second support arm at least partially below the leaflet.

In some examples, the method further comprises rotating a distal portion of the first delivery shaft relative to a proximal portion of the first delivery shaft to position the first support arm.

The one or more sutures may be configured to form loops extending at least partially through the leaflet. In some examples, the first support arm comprises a sheet supported by a wireframe.

In some examples, a distal portion of the sheet has a circular shape. A distal portion of the first support arm may be configured to extend generally perpendicularly relative to a proximal portion of the first support arm. The distal portion may be configured to naturally extend generally perpendicularly in response to removal from a support shaft. The first support arm may have a braided wire form.

### Additional Examples

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A delivery system comprising:
a first delivery shaft configured to deliver one or more sutures to a leaflet of a heart, the first delivery shaft having an end effector at a distal end of the first delivery shaft and configured to contact the leaflet;
a needle configured to extend from the distal end of the first delivery shaft and through the leaflet; and
a first support arm configured to contact and extend at least partially along the leaflet during delivery of the needle through the leaflet.

2. The delivery system of claim 1, wherein each of the one or more sutures is wrapped at least partially around a distal portion of the needle and is configured to form a knot anchor when removed from the needle.

3. The delivery system of claim 1, wherein the first support arm is configured to extend at least partially along an underside of the leaflet.

4. The delivery system of claim 3, further comprising a second support arm configured to extend at least partially along an upper side of the leaflet.

5. The delivery system of claim 1, further comprising a support shaft configured to deliver the first support arm.

6. The delivery system of claim 1, wherein the one or more sutures are configured to form loops extending at least partially through the leaflet.

7. The delivery system of claim 1, wherein the first support arm forms a loop with an opening.

8. The delivery system of claim 1, wherein the first support arm comprises a sheet supported by a wireframe.

9. The delivery system of claim 8, wherein a distal portion of the sheet has a circular shape.

10. The delivery system of claim 1, wherein the first support arm has a braided wire form.

11. A method for simulating treatment on a simulated patient in need thereof, the method comprising:
delivering a first delivery shaft to an underside of a leaflet of a heart, the first delivery shaft having an end effector at a distal end of the first delivery shaft, the first delivery shaft carrying a needle and one or more sutures;
extending a first support arm along the leaflet;
sliding the end effector towards an edge portion of the leaflet; and
extending the needle through the leaflet at the edge portion while the first support arm and the end effector are in contact with the leaflet.

12. The method of claim 11, wherein each of the one or more sutures is wrapped at least partially around a distal portion of the needle and is configured to form a knot anchor when removed from the needle.

13. The method of claim 11, further comprising extending the first support arm at least partially along an underside of the leaflet.

14. The method of claim 13, further comprising extending a second support arm at least partially along an upper side of the leaflet.

15. The method of claim 11, further comprising delivering a support shaft carrying the first support arm to the underside of the leaflet.

16. The method of claim 11, further comprising extending the first support arm at least partially above the leaflet.

17. The method of claim 16, further comprising lowering the first support arm to contact an upper side of the leaflet.

18. The method of claim 17, wherein the needle is extended through the leaflet while the leaflet is at least partially immobilized between the first support arm and the end effector.

19. The method of claim 16, wherein the needle is extending through an opening of the first support arm above the leaflet.

20. The method of claim 16, further comprising pressing the first support arm downward against the leaflet while pressing the end effector upward against the leaflet.
